(19) United States
(12) Patent Application Publication
(10) Pub. No.: US 2022/0387388 A1
(43) Pub. Date: Dec. 8, 2022

(54) SKIN-CARE BALM AND DEVICE FOR INTERNAL AND EXTERNAL NASAL APPLICATION

(71) Applicant: GSK CONSUMER HEAKTGCARE SARL, Prangins (CH)

(72) Inventor: Matina PAPAFRAGHA, Athens Lamia (GR)

(21) Appl. No.: 17/776,021

(22) PCT Filed: Nov. 12, 2020
(86) PCT No.: PCT/EP2020/081909

§ 371 Date: May 11, 2022

Related U.S. Application Data
(63) Continuation of application No. 15/930,847, filed on May 13, 2020.
Application No. 15/930,847 is a continuation-in-part of application No. 29/712,813, filed on Nov. 12, 2019.

(30) Foreign Application Priority Data
Nov. 14, 2019 (EP) 19209288.0
Nov. 6, 2020 (GR) 20200100669

(51) Int. Cl. A61K 31/4164; A61P 17/16; A61P 11/02; A61K 9/00; A61K 31/045; A61K 31/23; A61K 31/35; A61K 31/569; A61K 36/185; A61K 36/22; A61K 36/47; A61K 36/54; A61K 36/758; A61K 36/899

(52) U.S. Cl. CPC A61K 31/4164; A61P 17/16; A61P 11/02; A61K 9/0014; A61K 9/0043; A61K 31/045; A61K 31/23; A61K 31/35; A61K 31/569; A61K 36/185; A61K 36/22; A61K 36/47; A61K 36/54; A61K 36/758; A61K 36/899

(57) ABSTRACT

There is provided a skin-care balm comprising 15% to 45% (w/w) of two or more waxes, wherein a first wax has a melting point of 70° C. to 90° C. and a second wax has a melting point of 30° C. to 60° C., the balm further comprising 45% to 70% (w/w) of an emollient and a skin soothing agent. The skin-care balm can be applied to the skin in the perinasal area. There is also provided a combination product comprising a skin-care balm and a nasal spray product.

### CROSS-REFERENCE TO RELATED APPLICATIONS

This application claims benefit of European Patent Application 19209288.0 filed Nov. 14, 2019 and U.S. patent application Ser. No. 15/930,847 filed May 13, 2020, the disclosures of which are incorporated herein by reference in their entirety.

### FIELD OF THE INVENTION

This disclosure relates to skin-care balms. Specifically, it relates to a novel balm for the care of the skin around the nose.

### BACKGROUND TO THE INVENTION

The comfort, appearance, integrity and health of the skin in the perinasal area, including the upper lip area, can be impacted negatively by many factors. For example, patients suffering from conditions, such as nasal symptoms, regularly blow or clean their nose. This can lead to dryness, irritation, redness, soreness and the like of the perinasal skin. Nasal secretions can also irritate the perinasal skin, because of irritating ingredients and constant wetting of the perinasal area. Furthermore, nasal secretions or discomfort of the perinasal skin may trigger a patient to lick the affected perinasal area, which further dries out the skin and worsens the condition. Medicinal products and medicines, such as nasal sprays, are frequently used to treat nasal symptoms, but these products may also exacerbate irritation when they contact the perinasal skin.

Today, patients and consumers either leave this skin irritation untreated, treat it with skin creams or balms sold separately for different purposes, and/or conceal the skin irritation with make-up or other cosmetic products. However, existing cream or balm formulations in the market are not necessarily suitable for treating the irritation and redness around the nose. The use of concealer or make-up does not treat the condition and might even worsen it. Furthermore, existing skin-care balms are not adapted to application around the nose. For example, such balms often leave a greasy or shiny film on the skin which is not cosmetically desirable in the perinasal area. Additionally, these balms frequently have an unpleasant smell.

Furthermore, using a nasal spray and a separate product, such as a cream or balm, is not convenient, especially when both products need to be used several times per day. Users are commonly not at home throughout the day, but travel or commute to their workplace, school or other locations. Keeping track of multiple, separate products introduces undesirable complexity to user's routines.

Consequently, there remains a need for a skin-care balm that addresses one or more of the described influences specific to the perinasal skin, and which has desirable physical properties suitable for application to the area around the nose. There is also a need for a skin-care balm for the perinasal area that is suitable to prevent and treat discomfort, dry skin, feeling of tension, scaliness, flaking, redness, soreness and irritation. Finally, there also remains a need for a handy skin-care balm which can be applied to the perinasal skin comfortably.

### SUMMARY OF THE INVENTION

This disclosure provides a skin-care balm comprising 15%-45% (w/w) of two or more waxes, wherein a first wax has a melting point of 70° C.-90° C. and a second wax has a melting point of 30° C.-60° C., the balm further comprising 45%-70% (w/w) of an emollient and a skin soothing agent. In a preferred embodiment, the first wax is selected from rice bran wax, candelilla wax, bees wax, or any combination thereof. In another preferred embodiment, the first wax is rice bran wax.

In a preferred embodiment, the second wax comprises Rhus verniciflua peel cera, Rhus succedanea fruit cera, Shorea robusta resin, Euphorbia cerifera wax, or any combination thereof.

In another preferred embodiment, the emollient comprises octyldodecanol, caprylic/capric triglyceride, castor oil, avocado oil, cocoa butter, myristyl myristate, hydrogenated castor oil, shea butter, behenyl alcohol, cetyl esters, or any combination thereof. In another preferred embodiment, the emollient comprises 6%-20% (w/w) of botanical butter. In yet another preferred embodiment, the emollient comprises a vegetable oil comprising unsaturated fatty acids. In another preferred embodiment, the emollient comprises 6%-8% w/w of botanical butter which is cocoa butter and 4%-10% (w/w) of vegetable oil which is avocado oil. In yet another preferred embodiment the emollient comprises a powdered hydrogenated castor oil.

In a preferred embodiment, the skin soothing agent is Zanthoxylum bungeanum fruit extract.

In a preferred embodiment, the skin-care balm further comprises eucalyptol, a eucalyptus essential oil, menthol, menthyl ethylamido oxalate, or any combination thereof.

In a preferred embodiment, the balm has a melting point in the range from 60° C. to 75° C. In a more preferred embodiment, the melting point is in the range from 65° C. to 72° C.

In another preferred embodiment, the balm has a penetration value of 20 mm to 40 mm. More preferred is a penetration value of 25 mm to 35 mm.

In a preferred embodiment, the balm has a dome shape which is suitable to be applied directly to a perinasal area.

There is also provided a skin-care balm according to the disclosure, for use in the treatment of discomfort, dry skin, feeling of tension, scaliness, flaking, redness, soreness, and/or irritation of skin in a perinasal area. This disclosure further provides a method of treating a patient suffering from sore skin in the perinasal area comprising applying an effective amount of a skin-care balm according to the disclosure to skin in a perinasal area of a patient.

There is further provided a method for reducing skin redness in a perinasal area, the method comprising applying a skin-care balm described herein to the reddened skin.

There is further provided the use of a skin-care balm described herein in emolliating, hydrating, and/or calming of skin in a perinasal area. There is also provided the use of a skin-care balm described herein in a combination product comprising a nasal spray and said balm.

There is further provided a combination product comprising a nasal spray product and a skin-care balm described herein.

### Ingredients

### Waxes

Preferably, skin-care balm formulations comprise two or more waxes in a total amount of 15% to 45% by weight of the composition. Waxes have film forming properties and provide a water-impermeable layer when applied to the skin. This film protects the skin from external influences and retains physiological moisture. The use of two or more waxes lowers the melting point of the skin-care balm which eases application to the skin. This is especially useful for the skin-care balm of the present disclosure, as the skin-care balm can be applied directly to the skin of the user, without dosing and spreading with fingers. Due to this combination of waxes, the skin-care balm is soft enough in a wide range of temperatures to spread comfortably on the skin of the user, while also being solid enough to retain a defined shape at room temperatures. Thus an equilibrium is achieved between hardness and softness of the skin-care balm. The use of two or more waxes can also reduce the tendency of the waxes to form large crystals in some instances, which would make the balm grainy or brittle. This is important for the uniformity of the balm composition, and the feeling imparted upon balm contact with the skin during the application. Contact of the product with skin advantageously reduces or eliminates a sensation of friction or dryness, and allows formation of a homogeneous layer over the skin. This layer, or film, protects the skin from environmental factors, dryness and irritation.

It is preferred that a first wax has a melting point of 70° C. to 90° C. and a second wax has a melting point of 30° C. to 60° C. The combination of two waxes with these different melting points yields a base composition for the balm that has a suitable melting point for direct application to the skin. Details on suitable ranges of melting points are described below.

It was found that by controlling the drop point of a blend of two or more waxes to between 60° C. to 75° C., the resulting balm, including the emollient(s) and all other ingredients, strike the suitable equilibrium between hardness and softness. Therefore, the two or more waxes are combined, molten together, stirred and let harden. Thereafter, the drop point of this wax blend is determined. The drop point can be determined by the method monographed in the European Pharmacopoeia, Drop Point, Ph. Eur. Edition 10, method 2.2.17. It is preferred that a blend of the first and the second wax has a drop point in the range from 60° C. to 75° C. The resulting skin-care balm is soft, but mechanically resistant and retains a desired shape. This desired range of drop points of the wax blend can for example be achieved by combining a first wax and a second wax in a ratio of about 1:2 to 2:1 (w/w), wherein the first wax has a melting point of 70° C. to 90° C. and the second wax has a melting point of 30° C. to 60° C. Preferably, the ratio of first wax to second wax is about 1:1 (w/w).

In a preferred embodiment, the first wax comprises rice bran wax, candelilla wax, bees wax or any combination thereof. It has been found that with these waxes, the desired melting point and hardness-softness equilibrium are obtainable. Rice bran wax and candelilla wax are especially preferred, as these waxes are vegetal.

Preferably, the first wax is rice bran wax. Rice bran wax gives the balm formulation a matt finish and reduces shine and greasiness when applied to the skin. This is especially preferred as the skin-care balm is to be applied to the perinasal skin, and shiny facial skin appearance is not desirable cosmetically, as opposed to, for example, shine on the lips. Furthermore, rice bran wax is non-greasy and non-sticky, which is important for the skin feel of the product.

In a preferred embodiment, the second wax is Rhus verniciflua peel cera, Rhus succedanea fruit cera, Shorea robusta resin, Euphorbia cerifera wax, or any combination thereof. These waxes are preferred for having suitable melting points. These waxes are further preferred as they are vegetal waxes.

### Emollients

The balm further comprises emollients. The emollients hydrate the roughened skin of the perinasal area and soothe irritation. Furthermore, emollients lead to gelling of the wax component of the balm and thereby lower the balm's crystallisation tendency. This ultimately reduces the hardness of the balm, making spreading gentler, and ensuring the texture of the balm remains stable over time. Depending on the type of emollients used, the amount can be in the range from about 45% to 70% (w/w).

Any emollient known in the art can be used. Examples of suitable groups of emollients are botanical butters, vegetal oils, mono-, di- and triglycerides and mixtures thereof, fatty alcohols, fatly acids, and esters of fatty alcohols with fatty acids. Examples of suitable emollients are Butyrospermum parki (Shea) butter and Theobroma Cacao (Cocoa) seed butter, octyldodecanol, caprylic/capric triglyceride, Ricinus communis seed oil, hydrogenated castor oil, Persea gratissima (avocado) oil, isopropyl myristate, isocetyl stearate, myristyl myristate, isopropyl lanolate, cetyl esters, cetyl alcohol, stearyl alcohol, cetearyl alcohol, oleyl alcohol and behenyl alcohol.

In a preferred embodiment, the skin-care balm comprises octyldodecanol, caprylic/capric triglyceride, castor oil, avocado oil, cocoa butter, myristyl myristate, hydrogenated castor oil, shea butter, behenyl alcohol, cetyl esters, or a mixture of two or more thereof. It has been found that the use several different emollients is preferable to adjust the melting point and hardness properties of the skin-care balm. It was further found that the use of a botanical butter together with vegetable oils as emollients is especially preferable. This combination leads to a particularly pleasant skin feel of the final balm formulation. Furthermore, those ingredients are preferred as they are of vegetal origin.

In a preferred embodiment, the emollients comprise a powdered, hydrogenated castor oil. The powdered form of the hydrogenated castor oil reduces shine and greasiness of the balm when applied to the skin.

Furthermore, emollients comprising unsaturated fatty acids are preferred, for example shea butter and/or avocado oil. Avocado oil is especially preferred as it has a specific composition comprising free fatty acids that provides an excellent compatibility with the natural hydrolipidic film on the skin.

Preferably, the balm comprises 15%-45% (w/w) of two or more waxes, and 45%-70% (w/w) of emollients. In a preferred embodiment, the skin-care balm comprises 6% to 20% (w/w) of a botanical butter. In another preferred embodiment, the skin-care balm comprises 4% to 10% (w/w) of vegetable oil. In yet another preferred embodiment, the skin-care balm comprises 6% to 8% (w/w) of botanical butter which is cocoa butter and 4% to 10% (w/w) of vegetable oil which is avocado oil.

### Skin Soothing Agent

In some embodiments, the balm comprises skin soothing agent. The skin soothing agent has skin soothing properties, and can include ectoin, dexpanthenol, Aloe vera, calendula, bisabolol, niacinamide, or any combinations thereof. Alternatively, in other embodiments, the balm can comprise glycerine, hyaluronic acid, ceramides, phytosphingosine, cholesterol, madecassoside, bilberry seed oil, or a mixture of two or more thereof.

In a preferred embodiment, the skin soothing agent comprises an active ingredient that is a botanical extract. A botanical extract can be an extract from any part of a plant, e.g. an herbal extract. Botanical extracts are widely accepted by consumers, can be sourced environmentally friendly, and do not need to be chemically synthesized. Botanical extracts from edible plants are preferred as they are non-toxic and can be used in the perinasal and perioral area, such as the upper lip region, without limitation. The ingredients of the extracts can be controlled such that desired and effective ingredients are enriched, and undesirable ingredients are reduced or eliminated.

In another preferred embodiment, the skin soothing agent is Zanthoxylum bungeanum fruit husk extract. This extract is rich in lipophilic alkylamides such as hydroxy-alpha-sanshools and hydroxy-beta-sanshools. These agents act as agonists on receptors of sensory neurons, such as the transient receptor potential cation channel subfamily V member 1 TRPV1 (also known as capsaicin receptor, or vanilloid receptor) and the transient receptor potential cation channel subfamily A member 1, 1TRPA1. The interaction of the ingredients of the extract with the somatosensory neurons in the skin provides a soothing, numbing and anti-itching effect that is especially suitable for the balm described herein. The extract can provide a mild tingling effect and can give an immediate relief. The extract's soothing or paresthesial effect can relief the discomfort of the user, reduce the sensitivity of the skin, and reduce the body's response to irritations, such as a runny nose, tearing, and skin reddening. Zanthoxylum bungeanum fruit husk extract is not irritating to the skin and does not cause sensitisation. Additionally the extract has a pleasant smell that can mask any odour that might be inherent to further components of the balm. Zanthoxylum bungeanum fruit husk extract also has skin conditioning properties. The balm can therefore be formulated without perfume, which is preferred as perfumes can cause skin irritation or burning sensation, especially when used on damaged skin. Preferably, the Zanthoxylum bungeanum fruit husk extract is obtained by a mild, solvent free extraction method such as CO₂ extraction. This provides the advantage of a high yield of the lipophilic alkylamides with no residual organic solvents.

The Zanthoxylum bungeanum fruit husk extract can typically have a content of total alkylamides of 4% to 7% by weight. The Zanthoxylum bungeanum fruit husk extract can be used in the formulation of the balm in an amount from 0.05% to 1.0% by weight.

In an alternative embodiment, the skin soothing agent comprises Aloe barbadensis leaf extract. Aloe barbadensis leaf extract can have a moisturizing effect that is beneficial for roughened and cracked skin and helps to soothe irritated skin. Typically, the Aloe barbadensis leaf extract is used in the balm in an amount of 0.05% to 1% by weight.

### Further Ingredients

The balm can optionally comprise further excipients used in the art such as antioxidants, wetting agents, emulsifiers, solvents, matting agents, rheology additives, hydrating agents, and cooling agents.

Antioxidants can be used to enhance stability and shelf life. Antioxidants are especially preferred in embodiments comprising oils comprising unsaturated fatty acids. Examples of suitable antioxidants are butylhydroxytoluol (BHT) and vitamin E or derivatives thereof. Preferably, the antioxidants comprise tocopherol acetate which is a stable form of vitamin E and is known for its positive effects on the skin.

Inclusion of wetting agents can result in a balm that stays on the skin longer after application. Suitably, W/O emulsifiers can be used in some embodiments. Examples for suitable wetting agents are hydrophilic esters such as polyglyceryl-3-di-isostearate, cetylstearyl alcohol, PEG-30 dipolyhydroxystearate, or Lameform TGI.

An example of a suitable matting agent is talc, such as Talc LCW. Talc gives the balm a silky feeling and a good spreadability. An example of a suitable rheology additive is cosmedia Gel CC. It can improve the spreadability of the balm.

Examples for suitable cooling agents are essential oils, or synthetic or isolated individual constituents of essential oils. Other examples of suitable cooling agents are eucalyptol, eucalyptus essential oil, menthol, menthyl ethylamido oxalate, or a mixture of two or more thereof. The agents can have further beneficial functions in the formulation in some cases, such as perfuming, i.e. imparting a pleasant smell, masking the smell of other ingredients, skin conditioning, or soothing or refreshing properties.

The skilled artisan would appreciate that some ingredients may perform more than one role in a given formulation. For example certain ingredients may function as both skin emollient and emulsifier. As another example, waxes may have emollient properties. For example, cetearyl alcohol and myristyl myristate (listed herein under emollients) also are amphiphilic and can act as emulsifiers.

In one embodiment, the balm comprises the ingredients indicated in Table 1:

TABLE 1

| | % (w/w) of |
|---|---|
| | ingredient |
| | in the final balm |
| Ingredient type | formulation |
| Emollient(s) (oil, mono-, di- or triglyceride, | 30 to 45 |
| vegetal oil, or any combination thereof) | |
| Wax or pseudo wax, melting point in the range of | 5 to 25 |
| 70° to 90° C. (first wax) | |
| Wax or pseudo-wax, melting point in the range of | 5 to 25 |
| 30° C. to 60° C. (second wax) | |
| Emollient(s) (botanical butter(s)) | 5 to 30 |
| Emollient(s) (fatty alcohol, fatty acid, ester of fatty | 5 to 40 |
| alcohol with fatty acid, or any combination thereof) | |
| Emulsifier(s) | 0 to 5 |
| Skin soothing agent(s) | 0.05 to 2 |
| Antioxidant(s) | 0.05 to 1 |
| Perfume(s), essential oil(s) and/or cooling agent(s) | 0 to l |

In another embodiment, the balm comprises the ingredients indicated in Table 2:

TABLE 2

| | % (w/w) of |
|---|---|
| | ingredient |
| | in the final balm |
| Ingredient type | formulation |
| Octyldodecanol | 10 to 20 |
| Rice bran wax | 5 to 25 |
| Rhus verniciflua peel cera/ | 5 to 25 |
| Rhus sucedanea peel cera | |
| Emollient(s) (botanical butter(s)) | 5 to 15 |
| Emollient(s) (fatty alcohol, fatty acid, ester of fatty | 5 to 15 |
| alcohol with fatty acid, or any combination thereof) | |
| Emulsifier(s) | 1 to 5 |
| Skin soothing agent(s) | 0.05 to 2 |
| Antioxidant(s) | 0.05 to 1 |
| Perfume(s), essential oil(s) and/ | 0.01 to 1 |
| or cooling agent(s) | |

### Characteristics of the Balm

Preferably, the balm is a non-aqueous formulation. This reduces the risk of contamination of the balm upon contact with the skin and allows the use of a preservative-free formulation. This is beneficial as the balm can be used on irritated skin with an impaired barrier-function. Furthermore, a non-aqueous formulation forms a waterproof film or barrier layer on the irritated perinasal skin that has an occlusive effect, thus imparting a hydrating effect and protecting the skin from nasal discharge and repeated tissue wiping that typically exacerbates irritation of the skin. Such an occlusive film is furthermore preferred with the film serving as a substitute for a disrupted natural skin barrier in some cases, aiding in restoration of the natural skin barrier which prevents the skin from being damaged further in some instances, and reducing trans epidermal water loss in some cases. Furthermore, non-aqueous compositions have a mechanical strength that is preferred for form stability of the skin-care balm.

The skin-care balm may be referred to as solidified skin-care balm. Solidified, in the present disclosure, is to be understood as not liquid at 20° C. More preferred is that the balm is not liquid at 30° C., and most preferred is that the balm is not liquid at 40° C. Thereby, the skin-care balm retains the shape into which it is moulded over a wide range of common room temperatures.

Preferably, the melting temperature of the balm is in the range from 60° C. to 75° C. The melting point is determined according to the method monographed in the European Pharmacopoeia, Melting point open capillary method, Ph. Eur. Edition 10, method 2.2.15. Melting points in this range reduce the tendency of sweating or creaming of ingredients. This melting point range also provides firm adhesion of the balm to a balm base, as will be described in detail below, and structural integrity of the balm's shape, even in warm climates. At the same time, the balm is still soft enough for easy application to the skin and a convenient spread.

Preferably, the skin-care balm has a relative density at 20° C. (d₂₀²⁰) of 0.830 to 0.910. The relative density is measured according to the method monographed in the European Pharmacopoeia, Ph. Eur. Edition 10, method 2.2.5, using an automated density meter using an oscillating U-tube (e.g. Anton Paar).

In a preferred embodiment, the balm has a penetration value of 20 mm to 40 mm. More preferred is a penetration value of 25 mm to 35 mm. These ranges are preferred as higher penetration value indicates a too soft balm, which will impact the shape of the balm, as well as adhesion to a balm holder and the application properties of the balm. A too hard balm, indicated by a lower penetration value, will negatively impact the sensorial characteristics upon application. The penetration values are measured with the balm moulded in hemispherical mould, and are determined according to the method monographed in the European Pharmacopoeia, measurement of consistence by penetrometry, Ph. Eur. Edition 10, method 2.2.15.

Preferably, the balm has a form or shape that is suitably small at a free end or tapers towards a free end. By that, application in the small-scale area around the nose is made more easy. Preferably, the solidified balm has substantially a hemispherical shape, thus allowing for easy application. The hemispherical shape is also preferred because the circular outline can be combined with nasal spray products as described below under combination products. The balm is not limited to hemispherical shapes though, and can instead be any shape not inconsistent with the objectives of this disclosure. For example, in some embodiments, the balm can be in the form of a dome, stick, dermo-stick, cone, rounded cone, cylinder, pointed cylinder, oblong cylinder, pointed oblong cylinder, pyramid, or the like. A dome shape is to be understood as a shape that is a portion of a sphere cut off by a plane, not necessarily hemispherical. A dermo-stick is to be understood as having a shape similar to a lipstick.

The balm can be prepared by any methods known in the art that are not inconsistent with the objectives of this disclosure. For example, the solid (at room temperature) components can be provided in a pelleted, powdered, or shredded form, and can be combined and be molten together, e.g. in a water bath. Subsequently, the liquid (at room temperature) components can be added under stirring, and the mixture cooled under stirring. Lastly, after cooling but whilst the composition is still liquid, volatile agents can be added under stirring. When the mixture has a creamy composition, the mixture can be poured into suitable moulds, such as stainless steel moulds. Excess molten balm can be scratched off to provide an even surface in some cases. The balm can be cooled and hardened at room temperature or cooler, until solid. The solidified balm can then be removed from the moulds e.g. turning the mould upside down and tapping its edges on a hard surface. The balm can then be put into a balm holder element, which can be closed.

### Methods

The skin-care balm can be used for cosmetic methods. Therefore, there are provided in the present disclosure cosmetic methods of treatment. Provided are methods for reducing skin redness, for emolliating, hydrating and/or calming of skin and for reducing scaliness and/or roughness of skin in a perinasal area. There is also provided a method for reducing the external signs of a cold, comprising application of the skin-care balm of the present disclosure to the perinasal skin. There is provided a method for reducing skin redness in a perinasal area comprising application of the skin-care balm of the present disclosure to the reddened skin. The skin-care balm can be applied as often as needed. There is also provided a method for emolliating, hydrating and/or calming of skin in a perinasal area, comprising application of the skin-care balm of the present disclosure to the perinasal skin. The skin-care balm can be applied as often as needed. There is also provided a method for reducing scaliness and/or roughness of skin in a perinasal area, comprising application of the skin-care balm of the present disclosure to the perinasal skin. The skin-care balm can be applied as often as needed.

The skin care balm can also be used in methods of treatment of perinasal skin. It can be used in the treatment of discomfort, cracked skin, damaged skin, dry skin, feeling of tension, scaliness, flaking, redness, soreness and/or irritation. The present disclosure thus provides for methods of treatment of a patient suffering from discomfort, dry skin, feeling of tension, scaliness, flaking, redness, soreness and/or irritation of the skin in the perinasal area comprising applying an effective amount of a skin-care balm according to the present disclosure to skin in a perinasal area of the patient. The skin-care balm can be applied as often as needed. Alternatively, the skin-care balm can be applied two to three times daily. Optionally the skin-care balm can additionally be applied each time after cleaning or blowing the nose. An effective amount can be the amount of balm that forms a film on the treated areas of skin when the skin-care balm is applied to it.

The skin care balm can be for use in methods of treatment of perinasal skin. The methods of treatment can be treatment of discomfort, cracked skin, damaged skin, dry skin, feeling of tension, scaliness, flaking, redness, soreness and/or irritation. There is this disclosed a skin-care balm of the present disclosure for use in methods of treatment of discomfort, dry skin, feeling of tension, scaliness, flaking, redness, soreness and/or irritation of the skin in the perinasal area. The treatment can comprise applying an effective amount of a skin-care balm according to the present disclosure to skin in a perinasal area of the patient. The treatment can comprise applying the skin-care balm as often as needed. Alternatively, treatment comprises applying the skin-care balm two to three times daily. Optionally the skin-care balm can additionally be applied each time after cleaning or blowing the nose.

The present disclosure also provides new uses. There is provided the use of a skin-care balm of the present disclosure in emolliating, hydrating and/or calming of skin in a perinasal area. Furthermore, there is also provided the use of a skin-care balm of the present disclosure in a combination product comprising a nasal spray and said balm.

### Dosing/Application of the Balm

The balm can be applied to damaged, cracked, roughened, dry and irritated skin. It is especially beneficial in many embodiments for application in the perinasal area, i.e. the skin around the nose and the nostrils and the upper lip region. This area suffers primarily from wiping and nose blowing, nasal discharge, leakage of nasal formulation from the nose, and other effects of nasal symptoms. In some cases, the balm is beneficial for application to the perioral area and the lips, since the perioral area and the lips also suffer from the effects of nasal symptoms, such as leakage of nasal formulation or frequent licking.

According to a preferred embodiment, the balm is applied to the skin around the nose. In another preferred embodiment, the balm is applied to the skin around the nostrils. The skin-care balm can also be applied to the perioral area. The balm can be applied concomitantly with the product for nasal administration. Alternatively, the balm can be applied individually, when needed. For example, the balm can be applied after each cleaning of the nose or multiple times daily. The application is similar to the application of a dermo-stick or lipstick.

### Combination Product

There is also provided a combination product comprising a nasal spray product and a skin-care balm as described in the present disclosure. Preferably, the skin-care balm is contained in a balm holder element. Preferably, the balm holder element is located on a bottom of the nasal spray product. The combination product may be as described for example in European Patent Application 19209288.0, the entirety of which is incorporated by reference herein. The balm of the present disclosure is particularly suitable for those combination products due to its melting point, penetration, hardness/softness and other characteristics as described above. It firmly adheres to the balm holder element and it retains its desired shape in a wide range of temperatures.

In an alternative to the combination product, the skin-care balm can be provided in a balm holder element. It can be provided in a kit of parts together with a nasal spray product, or individually. Preferably, the balm holder element is suitable to be attached to a nasal spray product.

### DETAILED DESCRIPTION

### Definitions

The terms wax and waxes, as used herein, comprise waxes and pseudo-waxes. Numerous waxes or pseudo-waxes of animal, plant or petroleum origin known in the art are suitable for the formulation of the balm. Pseudo-waxes are excipients that have physical properties similar to those of waxes but are not waxes in the chemical sense, i.e. esters of fatty acids with fatty alcohols. They may be mixtures of hydrocarbons and/or fatty acids and/or fatty alcohols and/or fatty acid esters and/or hydrogenated or unhydrogenated oils (triglycerides). Examples for hydrocarbons are petrolatum and paraffin.

Skin conditioning agent may be any agent that has positive impact on skin and can be a pharmaceutical or cosmetic ingredient. Emollients are a group of skin conditioning agents that can act as lubricants on the skin or that maintain, restore, enhance and or balance skin hydration or moisture.

Unless indicated otherwise, the percentages given are weight percentages (w/w). INCI means the International Nomenclature of Cosmetic Ingredients. This is a system of names for waxes, oils, pigments, chemicals and other ingredients of cosmetic products and the likes. INCI ingredient names are predominantly based upon the terminology created by the INC committee.

Elements, apparatus, and methods described herein, however, are not limited to the specific embodiments presented in the detailed description, examples, and figures. It should be recognized that the exemplary embodiments herein are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

### Example

An example for a balm formulation is given below in Table 3.

TABLE 3
Example of a balm formulation
The names of the raw materials are given using the
International Nomenclature of Cosmetic Ingredients (INCI).
The % are indicated by weight.

| | % of | |
|---|---|---|
| | component in | |
| INCI name of | the final | Function of |
| component | formulation | the component |
| OCTYLDODECANOL | 17.00 | Emollient |
| CAPRYLIC/CAPRIC | 14.70 | Skin conditioning |
| TRIGLYCERIDE | | |
| RICINUS COMMUNIS | 14.30 | Emollient |
| SEED OIL | | Skin conditioning |
| ORYZA SATIVA CERA | 14.00 | Skin conditioning |
| | | Skin protecting |
| | | Smoothing |
| RHUS VERNICIFLUA | 13.40 | Emollient |
| PEEL CERA/RHUS | | Stabilising/ |
| SUCCEDANEA FRUIT | | Binding |
| CERA | | Emollient |
| THEOBROMA CACAO | 7.00 | Emollient |
| SEED BUTTER | | Skin conditioning |
| PERSEA GRATISSIMA | 5.00 | Skin conditioning |
| OIL | | |
| CETEARYL ALCOHOL | 3.00 | Emollient |
| | | Emulsifying |
| | | Emulsion stabilising |
| MYRISTYL MYRISTATE | 3.00 | Emollient |
| | | Skin conditioning |
| POLYGLYCERYL-3 | 2.50 | Emulsifying |
| DIISOSTEARATE | | |
| CETYL ESTERS | 2.00 | Emollient |
| | | Skin conditioning |
| BUTYROSPERMUM | 2.00 | Emollient |
| PARKII BUTTER | | Skin conditioning |
| OLEYL ALCOHOL | 0.48 | Emollient |
| | | Masking |
| ZANTHOXYLUM | 0.12 | Skin conditioning |
| BUNGEANUM FRUIT | | Skin soothing |
| EXTRACT | | |
| TOCOPHERYL ACETATE | 0.50 | Antioxidant |
| MENTHYL ETHYLAMIDO | 0.50 | Cooling |
| OXALATE | | Skin conditioning |
| Eucalyptol Nat | 0.2 | Cooling |
| | | Perfuming |
| | | Skin conditioning |
| MENTHOL | 0.20 | Cooling |
| | | Refreshing |
| | | Soothing |
| BHT | 0.10 | Antioxidant |

In this example, a balm was prepared by melting of the solid components in a water bath at a temperature sufficiently high to melt all ingredients, at about 60° C.-70° C. Subsequently, the liquid ingredients were added, and the mixture was stirred to form a bulk. Any volatile ingredients were added after letting the bulk cool under stirring, but whilst the bulk was still fluid. The fluid bulk was poured into a stainless steel mould with an array of recesses of hemispheric shape. Excess bulk was scraped off. The mould filled with the bulk was let cool down at 5° C. for 20 minutes. The solidified balm hemispheres were removed from the mould by turning it upside down and placing it on a hard surface. To produce the combination product, the balm was pressed on the base element (34, 34′) and the base element was assembled with the remaining parts of the balm holder element (30, 30″) and the container which contains the formulation for nasal administration.

1. A skin-care balm comprising 15% to 45% (w/w) of two or more waxes, wherein a first wax has a melting point of 70° C. to 90° C. and a second wax has a melting point of 30° C. to 60° C., the balm further comprising 45% to 70% (w/w) of an emollient and a skin soothing agent.

2. A skin-care balm according to claim 1, wherein the first wax comprises rice bran wax, candelilla wax, bees wax, or any combination thereof.

3. A skin care balm according to claim 1, wherein the first wax is rice bran wax.

4. A skin-care balm according to claim 1, wherein the second wax comprises Rhus verniciflua peel cera, Rhus succedanea fruit cera, Shorea robusta resin, Euphorbia cerifera wax, or a mixture of two or more thereof.

5. A skin-care balm according to claim 1, wherein the emollient comprises octyldodecanol, caprylic/capric triglyceride, castor oil, avocado oil, cocoa butter, myristyl myristate, hydrogenated castor oil, shea butter, behenyl alcohol, cetyl esters, or any combination thereof.

6. A skin-care balm according to claim 1, wherein the emollient comprises 6% to 20% (w/w) of botanical butter.

7. A skin-care balm according to claim 1, wherein the emollient comprises a vegetable oil comprising unsaturated fatty acids.

8. A skin-care balm according to claim 6, wherein the emollient comprises 6% to 8% (w/w) of botanical butter which is cocoa.

9. A composition according to claim 1, wherein the emollient comprises a powdered hydrogenated castor oil.

10. A skin-care balm according to claim 1, wherein the skin soothing agent is Zanthoxylum bungeanum fruit extract.

11. A skin-care balm according to claim 1, further comprising eucalyptol, a eucalyptus essential oil, menthol, menthyl ethylamido oxalate, or any combinations thereof.

12. A skin-care balm according to claim 1, wherein the balm has a melting point in the range from 60° C. to 75° C.

13. A skin-care balm according to claim 12, wherein the balm has a melting point in the range from 65° C. to 72° C.

14. A skin-care balm according to claim 1, wherein the balm has a penetration value of 20 mm-40 mm.

15. A skin-care balm according to claim 14, wherein the balm has a penetration value of 25 mm to 35 mm.

16. A skin care balm according to claim 1, wherein the balm has a dome shape suitable to be applied directly to a perinasal area.

17. A method for the treatment of discomfort, dry skin, feeling of tension, scaliness, flaking, redness, soreness and/or irritation of skin in a perinasal area including the step of applying the skin care balm according to claim 1.

18. A method of treating a patient suffering from sore skin in the perinasal area comprising applying an effective amount of a skin-care balm according to claim 1 to skin in a perinasal area of a patient.

19. A method for reducing skin redness in a perinasal area, the method comprising applying a skin-care balm according to claim 1 to the reddened skin.

20. A method of emolliating, hydrating and/or calming skin in a perinasal area including the step of applying the skin care balm according to claim 1.

21. A method of using the skin care balm according to claim 1 in a combination product comprising a nasal spray and the skin care balm including the step of applying the skin care balm from the combination product.

22. A combination product comprising a nasal spray product and a skin-care balm according to claim 1.

23. A skin-care balm according to claim 7, wherein the emollient comprises 4% to 10% (w/w) of a vegetable oil which is avocado oil.
